(19) United States
(12) Patent Application Publication
(10) Pub. No.: US 2004/0156803 A1
(43) Pub. Date: Aug. 12, 2004

(54) Cosmetic hair treatment process giving the hair long-lasting cosmetic properties

(76) Inventors: Gabin Vic, Venette (FR); Aude Livoreil, Paris (FR); Nicolas Daubresse, La Celle St Cloud (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/721,106

(22) Filed: Nov. 26, 2003

Related U.S. Application Data
(60) Provisional application No. 60/434,665, filed on Dec. 20, 2002.

(30) Foreign Application Priority Data
Nov. 29, 2002 (FR) 02 15076

(51) Int. Cl. A61K 7/06; A61K 7/11

(52) U.S. Cl. 424/70.11; 424/70.16

(57) ABSTRACT

The invention relates to a cosmetic hair treatment process comprising non-reducing activation of the hair, and then applying to the activated hair at least one cosmetically active compound capable of forming a covalent bond with the activated hair with the aid of one or more functional groups of the compound.

This application claims benefit of U.S. Provisional Application No. 60/434,665, filed Dec. 20, 2002.

Disclosed herein is a process for the cosmetic treatment of hair that may give the hair long-lasting cosmetic properties, for example, properties associated with resistance to shampooing, also referred to as “shampoo fastness.”

It is known in the art to treat hair by physicochemical absorption or fixing of a material onto the surface of the hair in order to increase the hair's sheen, to color the hair, or to make the hair more resistant to, for example, heat or light.

French patent FR 2 607 002 describes grafting a polyhaloacetyl polymer onto hair that has been previously reduced.

U.S. Pat. Nos. 4,973,475; 5,087,733; and 5,211,942 describe grafting thiol compounds onto hair that has been previously reduced.

U.S. Pat. No. 4,793,993 describes a process for treating keratin fibers, comprising reduction of the fibers followed by crosslinking of the fibers using diimidates or disuccinimidyl.

U.S. Pat. Nos. 5,300,285 and 5,270,036 describe a process comprising reducing hair and reacting the hair with a vinyl silicone.

Patent application WO 98/38974 describes a process for treating a keratin substrate, comprising reducing the disulphide linkages of keratin, followed by reacting the keratin with an active compound.

Moreover, it is also known in the art to treat hair by fixing material onto non-reduced hair. Thus, U.S. Pat. No. 5,523,080 describes grafting polymers bearing azlactone functions onto the hair.

Patent application WO 00/42010 describes applying onto the hair a cosmetic composition for grafting thiol compounds.

U.S. Pat. No. 5,935,560 describes applying to keratin fibers a composition for grafting a thiol silicone.

Patent application WO 01/00689 describes grafting compounds bearing dihydroxyacetone, N-hydroxysuccinimide, maleimide, phenylazide, benzophenone, amide, ester, diazo ester, diazo amide, imidate, α-halo ketone or pyridyl disulphide functional groups, or carbodiimide, vinyl sulphone, or isocyanate derivatives.

Patent application WO 00/59458 describes reacting a polymer comprising succinimide or glutimide functional groups on the hair.

However, these treatments may result in cosmetic properties that may not be sufficiently long-lasting, and that may, for example, show poor resistance to shampooing (“shampoo fastness”) since the material deposited may be rapidly eliminated by the action of water and surfactants.

The process disclosed herein proposes to overcome the drawbacks described above.

In particular, the present inventors have discovered, surprisingly, that a process comprising non-reducing activation of hair followed by fixing at least one cosmetically active compound onto the activated hair with the aid of at least one functional group of the at least one cosmetically active compound may make it possible to give the hair long-lasting cosmetic properties, for example shampoo-fastness properties.

As used herein, the term “shampoo fastness” means resistance to shampooing.

As used herein, the term “activation of the hair” means that the hair is subjected to a treatment that results in a change in the surface of the hair, such that at least one cosmetically active compound can then become fixed to the activated hair via at least one covalent bond. The activation may, for example, be physical and/or chemical.

As used herein, “non-reducing activation” means activation, in this case of the hair, without using any process that reduces the hair.

The present inventors have also discovered that hair thus treated may show long-lasting cosmetic properties, such as sheen, glint, softness, smoothness, suppleness or coloration, depending on the cosmetically active compound used.

The present inventors have further discovered that, on hair that has undergone degrading treatments beforehand, for example bleaching, or that has a damaged surface, the process disclosed herein may protect the surface of the hair and may give the hair a pleasant feel.

There may be other cosmetic effects associated with the process disclosed herein. These cosmetic effects may include, for example, color fastness, such as with respect to the stripping caused by washing, improvement of the shape hold, for example due to a change in the uptake of water or of moisture, and also increased anti-sun protection.

Other embodiments will become apparent on reading the description and the examples that follow.

One embodiment is thus a process for the cosmetic treatment of hair, comprising:

a) producing activated hair by non-reducing activation of the hair, and

b) applying to the activated hair at least one cosmetically active compound capable of forming at least one covalent bond with the activated hair with the aid of at least one functional group of the at least one cosmetically active compound, so as to graft the cosmetically active compound onto the activated hair.

The functional group(s) of the at least one cosmetically active compound may, for example, comprise at least one electrophilic center. In this manner, the formation of the at least one covalent bond may take place via the at least one electrophilic center.

The functional group(s) may, for example, be chosen from the following functional groups:

epoxides;

aziridines;

vinyls and activated vinyls, derived from acrylonitrile compounds, acrylic esters, methacrylic esters, crotbnic acids, crotonic esters, cinnamic acids, cinnamic esters, styrene and its derivatives, butadiene, vinyl ethers, vinyl ketones, and maleic esters;

carboxylic acids;

acetals;

hemiacetals;

disulphides;

aminals;

hemiaminals;

cyclic carbonates;

lactones;

thiolactones;

azlactones;

thioethers;

thiocyanates;

imines;

succinimides;

glutimides;

oxazines;

oxazolines;

ketones;

oxaziniums;

oxazoliniums;

aldehydes;

functional groups of the formula —RX, in which R is chosen from alkyl radicals, aryl radicals, and aralkyl radicals and X is chosen from alkyl, aryl and aralkyl halide functional groups, wherein the halide is chosen from I, Br, and Cl;

—OSO₃R′ in which R′ is chosen from hydrogen atoms and alkyl radicals;

—OSO₂R″ in which R″ is chosen from hydrogen atoms, alkyl radicals, and aryl radicals;

—N⁺(R′″)₃ in which R′″ is chosen from alkyl radicals and aryl radicals;

—OPO(OR″″)₂ in which R″″ is chosen from hydrogen atoms and alkyl radicals;

halide functional groups of at least one unsaturated ring, wherein the ring may, for example, be a carbon-based ring or a heterocycle of formula —R′″″X″, wherein R′″″ is chosen from unsaturated carbocyclic radicals and unsaturated heterocyclic radicals, and X″ is chosen from I, Br, and Cl. Mention may be made of, for example, chlorotriazine, chloropyrimidine, chloroquinoxaline and chlorobenzotriazole functional groups; and

sulphonyl halide functional groups of formula —SO₂X′, in which X′ is chosen from F and Cl.

In these illustrative functional groups, the formation of the at least one covalent bond with the activated hair may take place via the at least one electrophilic center of the at least one functional group.

The activation of the hair may, for example, be chosen from at least one of physical and chemical activation.

Physical activation of the hair may, for example, comprise subjecting the hair to at least one of heat, electromagnetic waves, electric fields, acoustic waves, and plasmas.

Chemical activation of the hair may for example comprise applying to the hair at least one compound capable of non-reducing activation of the hair.

The formation of the at least one covalent bond between the at least one cosmetically active compound and the activated hair may, for example, be obtained after at least one of a nucleophilic substitution reaction, electrophilic substitution reaction free-radical substitution reaction, addition reaction to carbon-carbon double or triple bonds, addition reaction to carbon-hetero atom double bonds or triple bonds, and ring-opening reaction.

The reaction between the at least one cosmetically active compound and the activated hair may, for example, take place spontaneously or the reaction may take place by activation through at least one of temperature, pH coreagents, and chemical catalysts.

The at least one cosmetically active compound may, for example, be chosen from simple molecules, polymers having only one functional group capable of forming a covalent bond with the activated hair, particles, and vesicles. The cosmetically active compound may, for example, be chosen from dye derivatives, sunscreens, vitamins, peptides, saccharides, agents known for moisturizing properties, such as glycerol, agents known for refreshing properties, such as menthol, and agents known for beneficial properties on the hair, such as panthenol.

Cosmetic properties of the hair that may be imparted by the cosmetically active compound may include, for example, softness and suppleness imparted by polydimethylsiloxane or dimethicone polyol, smoothness and softness imparted by polysaccharides such as guars, anti-soiling imparted by fluoro compounds, moisture resistance properties imparted by saturated linear alkyl chains comprising more than twelve carbon atoms, coloring and glint effects imparted by colored molecules, surface effects, such as friction or reliefs, optical effects imparted by particles, and beneficial effects imparted to degraded hair by molecules known for moisturizing or conditioning properties, such as glycerol, lanolin and fatty alcohols.

It may also be possible to use several cosmetically active compounds simultaneously, in order to impart several cosmetic properties simultaneously.

When the cosmetically active compound is a polymer, it may, for example, be synthesized via at least one of free-radical reactions, condensation reactions and ring-opening reactions. As used herein, the term “polymer” means a compound comprising at least five repeating units linked via covalent bonds.

The polymers synthesized via free-radical reaction may, for example, be chosen from polyacrylates, polymethacrylates, and polyvinyls.

The polymers synthesized via condensation reaction may, for example, be chosen from polyesters, polyethers, polyamides, polyurethanes, polydimethylsiloxane, and polypeptides.

The polymers synthesized via ring-opening reaction may, for example, be chosen from polyalkyleneimines and polyesters.

The polymers may also, for example, be chosen from polymers of natural origin, which may be optionally chemically modified. For example, the polymers of natural origin may be chosen from polysaccharides such as cellulose, dextran, chitosan, guar, and the hydroxyalkyl, carboxymethyl, amino and thiol derivatives thereof, as well as the derivatives thereof comprising aldehyde and epoxy functional groups.

The polymers may have any type of topology, i.e., shape. For example, they may be in linear, branched, starburst, or hyperbranched form, such as, for example, dendrimers.

When the cosmetically active compound is a polymer, the at least one functional group capable of forming at least one covalent bond with the activated hair of the cosmetically active compound may, for example, be present on the polymer chain, present at the end of a chain, grafted along the main chain, grafted on one of the side chains, present on a branch of the starburst, or present on a branch of the hyperbranched polymers.

Among the compounds capable of non-reducing activation of the hair, mention may be made of oxidizing agents, non-reducing polymers, acids, bases, non-reducing salts, and amphiphilic compounds.

Oxidizing agents may be chosen from, for example, aqueous hydrogen peroxide solution, bromates, and persalts.

Non-reducing polymers may be chosen from, for example, polyamines, polysaccharides, polyamides such as polypeptides and enzymes, (meth)acrylic acid homopolymers, and (meth)acrylic acid copolymers.

Acids may be chosen from, for example, hydrochloric acid, citric acid, and tartaric acid.

Bases may be chosen from, for example, sodium hydroxide, aqueous ammonia, and alkanolamines.

The non-reducing salt may be, for example, sodium chloride.

Amphiphilic compounds may be chosen from, for example, anionic, nonionic, amphoteric and cationic surfactants.

In certain embodiments, the compound capable of non-reducing activation of the hair may be chosen from oxidizing agents and non-reducing polymers. For example, the compound capable of non-reducing activation of the hair may be chosen from non-reducing polymers.

The compound capable of non-reducing activation of the hair may, for example, be capable of binding to the hair by hydrogen bonding or by adsorption.

The compound capable of non-reducing activation of the hair may be chosen, for example, from any compound capable of being adsorbed onto the hair in a long-lasting manner and having functional groups capable of reacting with the grafting functional groups of the cosmetically active compound. These functional groups of the compound capable of non-reducing activation of the hair may, for example, be chosen from hydroxyl, amine, and acid functional groups.

The compound capable of non-reducing activation of the hair may also, for example, be chosen from film-forming polymers having amine functional groups, which may naturally have good affinity for the hair, such as polyalkyleneimine, polyalkylamines, polylysines, polyallylamines, polyglutamines, hyperbranched polymers, dendrimers having amine end groups, and derivatives thereof.

Natural or modified polysaccharides, such as guar, dextran, cellulose and chitosan, may also constitute advantageous compounds capable of non-reducing activation of the hair, as they may already have cosmetic properties when applied to the hair.

The compound capable of non-reducing activation of the hair may also, for example, be chosen from proteins rich in amine or hydroxyl functional groups, hydrolysates and derivatives thereof, and particles that are insoluble but dispersible in a cosmetically acceptable medium and whose surface has reactive functional groups, for example by chemical modification of the particle's surface or by adsorption of a polymer having reactive functional groups onto the particle's surface.

The compound capable of non-reducing activation of the hair may also be chosen, for example, from vesicles such as molecular micelles and polymeric micelles.

The compound capable of non-reducing activation of the hair may, for example, be chosen from:

dendrimers whose chain ends are primary amines, for example polyamidoamines such as those sold under the tradename STARBURST® by the company Dendritech;

dendrimers or hyperbranched polymers having hydroxyl functional groups, such as those sold under the tradename NOVOLAK® by the company Clariant;

polyethyleneimines such as the product sold under the tradename LUPASOL® by the company BASF;

polylysines, such as the product sold under the tradename εPOLYLYSINE® by the company Chisso;

Hydroxy propyl celluloses, such as the product sold under the tradename KLUCEL EF® by the company Aqualon;

aminodextrans, such as the product sold by the company Carbomer,

polyvinyl alcohols;

polyvinyl amino alcohols, such as the product sold by the company Carbomer,

chitosans;

carboxymethyldextrans, such as the product sold by the company Fluka; and

carboxymethylchitosans, such as the product sold under the tradename OLEVASAN® by the company Sino Lion.

Another embodiment may comprise a composition comprising at least one compound capable of non-reducing activation of the hair and at least one cosmetically active compound. Examples of this composition may be chosen, for example, from the following compositions:

polyethyleneimine/fluorescent derivative comprising at least one group chosen from —SO₂Cl, chlorotriazine, and thiolactone groups;

polyethyleneimine/polydimethylsiloxane having at least one monofunctional epoxy group; and

chitosan/sugars in aldehyde form.

It is also possible to use more than one compound capable of non-reducing activation of the hair.

The process for the cosmetic treatment of hair disclosed herein generally comprises the following:

a) producing activated hair either by treating the hair using a physical activation process for a time sufficient to activate the hair, for example a time ranging from 1 to 60 minutes, or applying to the hair for a leave-in time ranging from 1 to 60 minutes at least one compound capable of non-reducing activation of the hair, optionally under the application of heat;

b) and then after optionally rinsing, applying to the activated hair, for a leave-in time ranging from 1 to 60 minutes, at least one cosmetically active compound capable of forming at least one covalent bond with the activated hair.

The examples that follow are intended to illustrate the invention.

### EXAMPLE 1

### Grafting of a Cosmetically Active Compound onto a Polyethyleneimine (PEI) Coating

Solutions used:

| Solution A | |
|---|---|
| Polyethyleneimine (PM25000) sold under the | 10 g |
| tradename LUPASOL ® by the company BASF | |
| 36% HCl | qs pH 8 |
| H₂O | qs 100 g |

| Solution B | |
|---|---|
| Reactive Blue 4 dye | 5 g |
| H₂O | qs 100 g |

The Reactive Blue 4 dye is a dye comprising a chlorotriazine functional group, having the following formula:

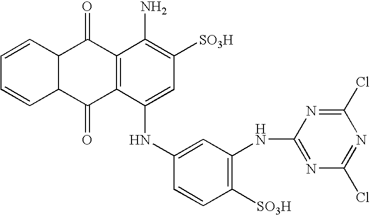

0.4 g of solution A was applied to locks (2.5 g) of hair. The locks were maintained at 60° C. for 30 minutes. Without intermediate rinsing, 0.4 g of solution B was applied to the locks. The locks were then maintained at 30° C. for 30 minutes.

The locks were then rinsed with water and dried. Colored locks were obtained with more intense color than that obtained with locks treated with solution B only. This color was also resistant to shampooing.

### EXAMPLE 2

### Grafting of Glucose onto a Polyethyleneimine (PEI) Coating

Solutions used:

| Solution A | |
|---|---|
| Polyethyleneimine (PM75000) sold under the | 5 g |
| tradename LUPASOL ® by the company BASF | |
| H₂O | qs 100 g |

| Solution B | |
|---|---|
| Glucose | 5 g |
| H₂O | qs 100 g |

0.4 g of solution A was applied to locks (2.5 g) of hair. The locks were maintained at 60° C. for 30 minutes. Without intermediate rinsing, 20 ml of solution B was applied to the locks. 0.12 g of NaBH₃CN was then added. The locks were then maintained at room temperature for 16 hours.

The locks were then washed with shampoo, rinsed with water, and dried. Colored locks having increased mass and volume when wet and pronounced softness when dry were obtained. These effects also resistant to shampooing.

What is claimed is:

1. A process for the cosmetic treatment of hair, comprising:
a) producing activated hair by non-reducing activation of the hair; and
b) applying to the activated hair at least one cosmetically active compound comprising at least one functional group capable of forming at least one covalent bond with the activated hair.

2. The process according to claim 1, wherein the at least one functional group of the cosmetically active compound comprises at least one electrophilic center.

3. The process according to claim 1, wherein the at least one functional group of the cosmetically active compound is chosen from the following functional groups:
epoxides;
aziridines;
vinyls and activated vinyls, derived from acrylonitrile compounds, acrylic esters, methacrylic esters, crotonic acids, crotonic esters, cinnamic acids, cinnamic esters, styrene and its derivatives, butadiene, vinyl ethers, vinyl ketones, and maleic esters;
carboxylic acids;
acetals;
hemiacetals,
disulphides;
aminals
hemiaminals;
cyclic carbonates;
lactones;
thiolactones;
azlactones;
thioethers;
thiocyanates;
imines;
succinimides;
glutimides;
oxazines;
oxazolines;
ketones;
oxaziniums;
oxazoliniums;
aldehydes;
functional groups of the formula —RX, in which R is chosen from alkyl radicals, aryl radicals, and aralkyl radicals and X is chosen from I, Br, and Cl;
—OSO₃R′ in which R′ is chosen from a hydrogen atom and from alkyl radicals;
—OSO₂R″ in which R″ is chosen from a hydrogen atom, from alkyl radicals, and from aryl radicals;
—N⁺(R′″)₃ in which R′″ is chosen from alkyl radicals and aryl radicals;
—OPO(OR″″)₂ in which R″″ is chosen from a hydrogen atom and from alkyl radicals;
halide functional groups of at least one unsaturated ring; and
functional groups of the formula —SO₂X′, in which X′ is chosen from F and Cl.

4. The process according to claim 3, wherein the at least one unsaturated ring is chosen from carbon-based rings and heterocycles of the formula —R′″″, wherein R′″″ is chosen from unsaturated carboxylic radicals and unsaturated heterocyclic radicals and X″ is chosen from I, Br, and Cl.

5. The process according to claim 1, wherein the non-reducing activation of hair is. chosen from at least one of physical activation and chemical activation.

6. The process according to claim 5, wherein the non-reducing activation of hair is physical activation.

7. The process according to claim 6, wherein the physical activation comprises subjecting the hair to at least one of heat, electromagnetic waves, electric fields, acoustic waves, and plasmas.

8. The process according to claim 5, wherein the non-reducing activation of hair is chemical activation.

9. The process according to claim 8, wherein the chemical activation comprises applying to the hair at least one compound capable of non-reducing activation of hair.

10. The process according to claim 1, wherein the formation of the at least one covalent bond between the cosmetically active compound and the activated hair is obtained after at least one reaction chosen from a nucleophilic substitution reactions, an electrophilic substitution reaction, a free-radical substitution reaction, an addition reaction to carbon-carbon or carbon-hetero atom double bonds, an addition reaction to carbon-carbon or carbon-hetero atom triple bonds, and a ring-opening reaction.

11. The process according to claim 1, wherein the formation of the at least one covalent bond takes place spontaneously.

12. The process according to claim 1, wherein the formation of the at least one covalent bond takes place by activation through at least one of temperature, pH, coreagents, and chemical catalysts.

13. The process according to claim 1, wherein the cosmetically active compound is chosen from simple molecules, polymers having only one functional group capable of forming a covalent bond with the activated hair, particles, and vesicles.

14. The process according to claim 13, wherein the cosmetically active compound is chosen from dye derivatives, sunscreens, vitamins, peptides, saccharide, moisturizing agents, refreshing agents, and agents known for beneficial properties on hair.

15. The process according to claim 13, wherein the cosmetically active compound comprises at least one polymer synthesized via at least one reaction chosen from a free-radical reaction, a condensation reaction, and a ring-opening reaction.

16. The process according to claim 15, wherein the at least one polymer synthesized via at least one free-radical reaction is chosen from polyacrylates, polymethacrylates, and polyvinyls.

17. The process according to claim 15, wherein the at least one polymer synthesized via at least one condensation reaction is chosen from polyesters, polyethers, polyamides, polyurethanes, polydimethylsiloxane, and polypeptides.

18. The process according to claim 15, wherein the at least one polymer synthesized via at least one ring-opening reaction is chosen from polyalkyleneimines and polyesters.

19. The process according to claim 13, wherein the polymers are chosen from polymers of natural origin and are optionally chemically modified.

20. The process according to claim 19, wherein the polymers are chosen from cellulose, dextran, chitosan, guar, hydroxyalkyl derivatives thereof, carboxymethyl derivatives thereof, amino derivatives thereof, thiol derivatives thereof, and derivatives thereof containing at least one of aldehyde and epoxy functional groups.

21. The process according to claim 13, wherein the polymers are in linear,. branched, starburst, or hyperbranched form.

22. The process according to claim 9, wherein the compound capable of non-reducing activation of hair is capable of binding to the hair by at least one of hydrogen bonding and adsorption.

23. The process according to claim 9, wherein the compound capable of non-reducing activation of hair is chosen from oxidizing agents, non-reducing polymers, acids, bases, non-reducing salts, and amphiphilic compounds.

24. The process according to claim 23, wherein the oxidizing agent is chosen from aqueous hydrogen peroxide solution, bromates, and persalts.

25. The process according to claim 23, wherein the non-reducing polymers are chosen from polyamines, polyamides, (meth)acrylic acid homopolymers, and (meth)acrylic acid copolymers.

26. The process according to claim 23, wherein the acid is chosen from hydrochloric acid, citric acid, and tartaric acid.

27. The process according to claim 23, wherein the base is chosen from sodium hydroxide, aqueous ammonia, and alkanolamines.

28. The process according to claim 23, wherein the non-reducing salt is sodium chloride.

29. The process according to claim 23, wherein the amphiphilic compounds are chosen from anionic, non-ionic, amphoteric, and cationic surfactants.

30. The process according to claim 9, wherein the compound capable of non-reducing activation of hair is chosen from polyalkyleneimines and derivatives thereof, polyalkylamines, polylysines, polyallylamines, polyglutamines, hyperbranched polymers, dendrimers comprising amine end groups and derivatives thereof, natural polysaccharides, modified polysaccharides, proteins comprising at least one amine functional group, proteins comprising at least one hydroxyl functional group, hydrolysates and derivatives thereof, particles that are insoluble but dispersible in a cosmetically acceptable medium and whose surface comprises at least one reactive function, and vesicles.

31. The process according to claim 30, wherein the vesicles are chosen from molecular micelles and polymeric micelles.

32. The process according to claim 9, wherein the compound capable of non-reducing activation of hair is chosen from:
dendrimers whose chain ends are primary amines;
dendrimers comprising at least one hydroxyl functional group;
hyperbranched polymers comprising at least one hydroxyl functional group;
polyethyleneimines;
polylysines;
hydroxypropyl cellulose;
aminodextran;
polyvinyl alcohols;
polyvinyl amino alcohols;
chitosan;
carboxymethyldextran; and
carboxymethylchitosan.

33. A process for the cosmetic treatment of hair, comprising:
a) producing activated hair by treating the hair using a physical activation process for a time sufficient to activate the hair, optionally under heat; and
b) after optionally rinsing, applying to the activated hair for a leave-in time ranging from 1 to 60 minutes at least one cosmetically active compound capable of forming at least one covalent bond with the activated hair.

34. The process according to claim 33, wherein said time sufficient to activate the hair is a leave-in time ranging from 1 to 60 minutes.

35. A process for the cosmetic treatment of hair, comprising:
a) producing activated hair by applying to the hair for a leave-in time ranging from 1 to 60 minutes at least one compound capable of non-reducing activation of the hair, optionally under heat; and
b) after optionally rinsing, applying to the activated hair for a leave-in time ranging from 1 to 60 minutes at least one cosmetically active compound capable of forming at least one covalent bond with the activated hair.
